(19) United States
(12) Patent Application Publication
(10) Pub. No.: US 2024/0207389 A1
(43) Pub. Date: Jun. 27, 2024

(54) METHODS FOR IMPROVING COVID VACCINE IMMUNOGENICITY

(71) Applicant: Northwestern University, Evanston, IL (US)

(72) Inventor: Pablo Penaloza-MacMaster, Evanston, IL (US)

(21) Appl. No.: 18/308,349

(22) Filed: Apr. 27, 2023

Related U.S. Application Data
(60) Provisional application No. 63/335,519, filed on Apr. 27, 2022.

(51) Int. Cl. A61K 39/215; A61K 39/00; A61P 37/04

(52) U.S. Cl. CPC A61K 39/215; A61P 37/04; A61K 2039/53; A61K 2039/545; A61K 2039/575

(57) ABSTRACT

The present disclosure provides an improved vaccine compositions and methods for eliciting an immune response against SARS-COV-2 and providing broader protection against SARS-COV-2 variants.

### CROSS-REFERENCE TO RELATED APPLICATIONS

This application claims priority to U.S. Provisional Application No. 63/335,519 filed on Apr. 27, 2022, the content of which is incorporated by reference in its entirety.

### STATEMENT REGARDING FEDERALLY SPONSORED RESEARCH

This invention was made with government support under grant number DA051912 awarded by the National Institutes of Health. The government has certain rights in the invention.

### REFERENCE TO AN ELECTRONIC SEQUENCE LISTING

The contents of the electronic sequence listing (702581.02338.xml; Size: 12.01 bytes; and Date of Creation: Apr. 21, 2023) is herein incorporated by reference in its entirety.

### BACKGROUND

Coronaviruses (CoV) constitute a large family of positive-stranded, enveloped RNA viruses that infect a broad range of mammalian and avian species. These viruses cause primarily respiratory and enteric diseases. In the last two decades three new zoonotic CoVs have emerged to infect humans. The most recent emergence of SARS-COV-2 that continues to spread globally raises many scientific and public health questions and challenges. Development of effective vaccines and antiviral therapeutics and rapid deployment of both is a pressing need. This will be an even more critical priority if SARS-COV-2 continues to spread and becomes endemic in the respiratory virus disease landscape. Previous work with the other two recent emergent pathogenic human CoVs, severe acute respiratory syndrome (SARS-COV) and Middle East respiratory syndrome (MERS-COV), provides insight and platforms that can help expedite the process, but none of these have moved beyond early trial stages. Much remains to be learned about the SARS-CoV-2 and its interplay with its human host and what will constitute the most effective, safe vaccine strategy. There are currently seven CoVs that infect humans, HCoVs OC43, 229E, NL63 and HKU1, that cause seasonal upper respiratory infections, in addition to the three more pathogenic viruses. The human viruses are thought to have emerged from zoonotic hosts to infect humans. Viral genomic analyses indicate that the human viruses are related to bat CoVs. A large number of novel CoVs have been identified in bat populations since identification of SARS-COV and the expectation is that we will continue to have spillover of these viruses to humans. This reinforces the need for development of vaccines against emergent CoVs.

### SUMMARY

The present invention provides a vaccine composition capable of eliciting an immune response in a subject against SARS-COV-2, the vaccine composition comprising a mutant SARS-Cov-2 spike protein comprising D614G mutation (SEQ ID NO:1) or a portion thereof; or an mRNA encoding the mutant SARS-Cov-2 spike protein or portion thereof. In an embodiment, the portion of the mutant comprises SEQ ID NO: 6. In an embodiment, the vaccine composition further comprises a nanoparticle. In an embodiment, the nanoparticle encapsulates the protein. In an embodiment, the nanoparticle is a lipid nanoparticle.

The present invention also provides a method of eliciting an immune response to a SARS-CoV-2 spike protein in a subject, the method comprising administering to the subject the vaccine composition described herein in an amount effective to elicit the immune response. In an embodiment, the immune response is a humoral immune response. In an embodiment, the humoral immune response comprises an increase in the antibody titers against SARS-COV-2 spike protein. In an embodiment, the administering comprises administering the vaccine composition at least two times. In an embodiment the time between the first and second administration is at least about two weeks. In an embodiment, the time between the first and second administration is between about two and about three weeks. In an embodiment, the method comprises administering the vaccine intramuscularly. In an embodiment, the subject is a human.

The present invention also provides a method of eliciting a long-term immune response to a SARS-COV-2 spike protein in a subject, the method comprising administering to the subject the vaccine composition described herein in an amount effective to elicit the immune response for longer than six months from the time of administration. In an embodiment, the administering comprises administering the vaccine at least two times. In an embodiment, the time between the first and second administration is at least about two weeks. In an embodiment, the time between the first and second administration is between about two and about three weeks. In an embodiment, the immune response lasts longer than about 12 months from the time of administration. In an embodiment, the method comprises administering the vaccine intramuscularly. In an embodiment, the subject is a human.

### BRIEF DESCRIPTION OF THE DRAWINGS

FIGS. 1A-1B. The D614G Mutation in SARS-COV-2 Spike Protein. A) Diagram of SARS-CoV-2 depicting the spike, nucleocapsid, membrane, envelope and RNA viral genome; and crystal structure of SARS-COV-2 spike protein. B) Schematic representation of the SARS-COV-2 S protein. Abbreviations: NTD— N terminal domain; RBD— receptor binding domain; HR 1/2—heptad repeat 1/2; TM— transmembrane domain; CT— cytoplasmic tail; S1/S2— subunit 1/2. Zhang et al. Trends in Genetics, April 2021, Vol. 37, No. 4.

FIGS. 2A-2B. D614G-based mRNA vaccine is more immunogenic than ancestral mRNA vaccines. Mice were immunized with 3 ug of either an ancestral-based mRNA vaccine or with an D614Gbased mRNA vaccine, and SARS-COV-2 RBD-specific IgG titer antibody responses were measured against A) ancestral-specific SARS-COV-2 spike or B) Omicron variant-specific SARS-CoV-2 spike.

### DETAILED DESCRIPTION

The present disclosure describes severe acute respiratory syndrome coronavirus 2 (SARS-COV-2) vaccine compositions, as well as methods for making and using the same that result in an enhanced immune response.

Severe acute respiratory syndrome coronavirus 2 (SARS-COV-2) is the third highly pathogenic human CoV to emerge in the past two decades. The virus causes COVID-19, a severe respiratory disease with an estimated mortality of 2-3% that rapidly spread across China beginning in late 2019 and was declared a world-wide pandemic in early 2020. Like other CoVs, the spike (S) protein is assumed to be the major target for neutralizing antibodies. SARS-COV-2 S protein binds to the receptor, angiotensin-converting enzyme 2 (ACE2), through its receptor binding domain (RBD). The RBDs for other CoVs are immunogenic and a major neutralizing determinant. There are significant concerns that SARS-COV-2 will become embedded in the viral respiratory disease landscape that will be encountered seasonally. Thus, development of additional safe and effective vaccines against the virus is a significant priority. The present disclosure provides a vaccine that is safe, efficacious and durable against SARS-COV-2. Standard molecular biology, biochemical approaches, vaccination and immunogenicity assessments were used to generate a SARS-COV-2 mRNA vaccine, specifically a SARS-COV-2 D614G mRNA vaccine. This vaccine was evaluated for immune responses elicited in mice.

Current vaccines based on the ancestral (Wuhan) strain of SARS-COV-2 are effective at preventing severe illness and death, but immune responses wane within months, necessitating subsequent boosters to keep the immune response high. The present disclosure's D614G-based mRNA vaccine elicits high and durable responses that may obviate the need for further boosters. This mutant vaccine also seems to better recognize SARS-COV-2 variants, relative to the ancestral vaccine, providing better broad-spectrum coverage than the current vaccines.

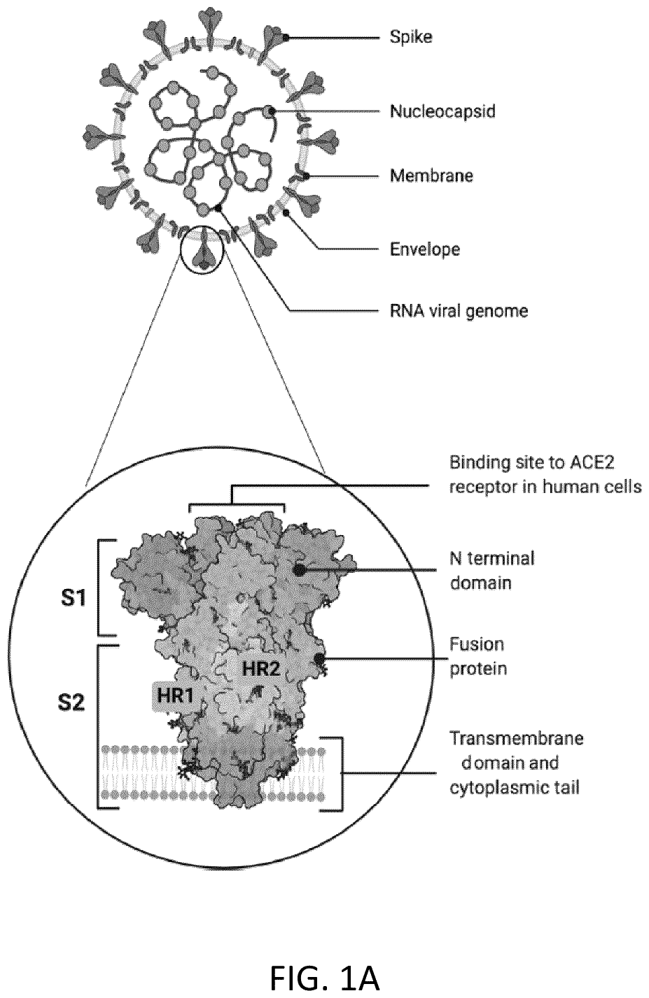

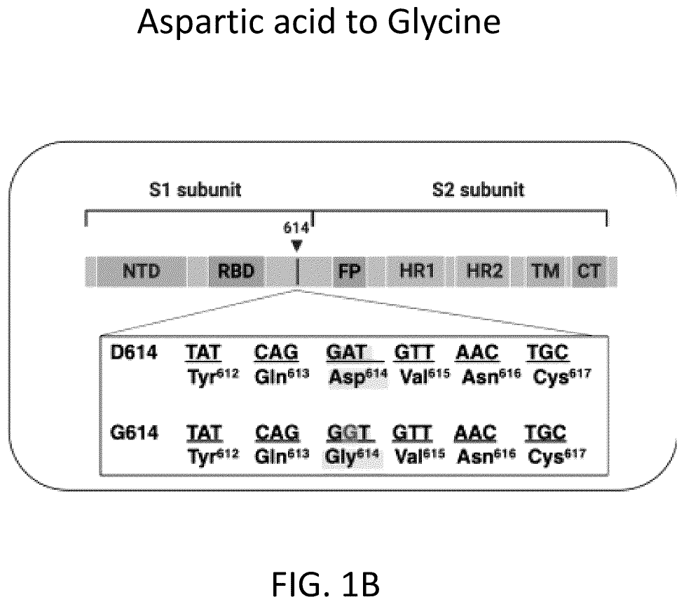

SARS-COV-2 includes membrane (M), spike (S), envelope (E), and nucleocapsid (N) structural proteins. The M, S, and E proteins provide the structure of the exterior viral envelope. The S protein is a glycoprotein that mediates receptor binding and fusion during entry into a host cell. A mutated portion of the spike (S) protein of SARS-COV-2 used in the present invention has a D614G amino acid mutation (SEQ ID NO: 4 (genomic), SEQ ID NO: 6 (protein)). This missense mutation is caused by an A-to-G nucleotide mutation at position 23,403 in the ancestral Wuhan reference strain and results in an aspartate to glycine amino acid change at position 614 (D614G), as illustrated in FIG. 1C (Zhang et al. Trends in Genetics, April 2021, Vol. 37, No. 4).

### Compositions

In a first aspect, the disclosure provides a vaccine composition capable of eliciting an immune response in a subject against SARS-COV-2. The vaccine composition comprises or consists of an mRNA encoding mutant SARS-COV-2 spike protein comprising D614G mutation, or a mutant SARS-COV-2 spike protein comprising D614G mutation. The SARS-COV-2 spike protein sequence with D614G mutation (D614G mutant) may comprise SEQ ID NO: 1 or a sequence having at least 95% identity to SEQ ID NO: 1 and comprising the D614G mutation. A polynucleotide sequence encoding the D614G mutant may comprise SEQ ID NO: 2 or a sequence having at least 95% identity to SEQ ID NO: 2 and encoding the D614 mutation.

In a second aspect, the disclosure provides a vaccine composition comprising or consisting of an mRNA encoding a mutated portion of the spike protein of SARS-COV-2 having a D614G amino acid mutation, the mutated portion comprising SEQ ID NO: 6 or a sequence having at least 95% identity to SEQ ID NO: 6. A polynucleotide sequence encoding the portion of the D614G mutant may comprise SEQ ID NO: 4 or a sequence having at least 95% identity to SEQ ID NO: 4 and encoding the D614 mutation.

The vaccine composition described herein is capable of eliciting or generating an immune response. The term “subject” may be used interchangeably with the terms “individual” and “patient” and includes human and non-human mammalian subjects. The term “subject” does not denote a particular age or sex. In an embodiment, the subject is a human. The subject may be infected with, suspected of being infected with or at risk for infection with a coronavirus. In some embodiments the subject is infected with SARS-COV-2, is suspected of being infected with SARS-CoV-2, or is at risk of becoming infected with SARS-COV-2.

Eliciting or generating an immune response refers to the activation of any component of the immune system in a subject. In some embodiments, generating an immune response includes a humoral immune response. Humoral immunity produces antigen-specific antibodies and is primarily driven by B cells. Humoral immune responses comprise the production of antibodies, complement proteins, and antimicrobial peptides. An immune response generated by the methods and compositions provided herein may further comprise the production of immune mediators including, but not limited to cytokines (IFNγ, IL-1β, TNF-α, IL-6, IL-21, IL-4, IL-5, IL-9, IL-17, IL-12, IL-2), chemokines and cytotoxic granules. An immune response generated by the methods and compositions provided herein may further comprise the production, activation and/or maturation of other immune cells including, but not limited to macrophages, dendritic cells, B lymphocytes, T lymphocytes, neutrophils, monocytes and plasma cells

The vaccine compositions described herein may be able to recognize variants of concern (VOC). A VOC is a category used for variants of SARS-COV-2 where mutations in the spike protein receptor binding domain (RBD) substantially increase binding affinity in the RBD-hACE2 complex, and are linked to rapid spread in human populations (epidemiological data). The compositions described herein may also be better able to recognize variants of interest (VOI) or variants under investigation (VUI). In embodiments, the vaccine compositions described herein recognize VOC including, but not limited to Alpha, Beta, Gamma, Delta, Epsilon, Eta, Iota, Kappa, Omicron, Zeta, Mu and their lineages and descendant lineages. In particular embodiments, the vaccine compositions described herein recognize the VOC Omicron and subsequent lineages.

The vaccine composition may comprise or consist of the D614G mutant and a pharmaceutically acceptable carrier.

The vaccine composition may further comprise a nanoparticle capable of encapsulating the D614G mutant or mRNA encoding the D614G mutant or incorporating the D614G mutant within its membrane, such that the mutant spike is on the surface of the nanoparticle. A “nanoparticle” or “nanocarrier” is a nanomaterial used as a transport module for another substance, such as a polynucleotide or protein. Nanoparticles include micelles, polymers, carbon-based materials, dendrimers, and polymeric or lipid-based carriers, such as liposomes or lipid nanoparticles. In preferred embodiments, the nanoparticles are lipid nanoparticles. A nanoparticle as used herein is typically has at least one dimension in the 1-500 nanometer scale. Suitable lipid nanoparticles are known in the art and include, for example, a combination of a lipid, choline (e.g., distearoylphosphatidylcholine (DSPC)), cholesterol, and combinations thereof. Additionally, the nanoparticles may further comprise a stabilizer. For example, suitable nanoparticles are the Gen Voy ILM™ (Precision Nanosystems). The Gen Voy-ILM™ is an ionizable lipid mix that enables the rapid and easy production of RNA-loaded lipid nanoparticles (LNPs) using the NanoAssemblr®Platform (Percision Nanosystems). Lipid nanoparticles are combined with the D614G mutant or mRNA encoding the D614G mutant in ratios that optimize encapsulation efficiency and biological activity, for example as with the Gen Voy ILM™ system.

As used herein “vaccine” refers to a composition that includes an antigen or a nucleotide encoding an antigen. A vaccine may also include a biological preparation that improves immunity to a particular disease. An antigen may resemble a disease-causing pathogen or portions thereof, and may be made from weakened or killed forms of the pathogen, its toxins or one of its surface proteins. In the vaccine compositions disclosed herein, the antigen is SARS-COV-2 spike protein with a D614G mutation. The antigen stimulates the body's immune system to recognize the pathogen as foreign, destroy it, and “remember” it, so that the immune system can more easily recognize and destroy the pathogen if it encounters it later.

Vaccines may be prophylactic, e.g., to prevent or ameliorate the effects of a future infection by a natural pathogen, or therapeutic, e.g., to treat the disease incurred as a result of infection by the pathogen. Administration of the vaccine to a subject results in an immune response, generally against the pathogen. The amount of vaccine that is therapeutically effective may vary depending on the particular pathogen or the condition of the subject. The vaccine may be introduced directly into the subject by parenteral or oral administration, intramuscular injection, subcutaneous/intradermal injection, intravenous injection, intrathecal administration, buccal administration, transdermal delivery, topical administration, intranasal administration, respiratory administration, etc.

The vaccine compositions described herein may also include a suitable carrier or vehicle for delivery. As used herein, the term “carrier” refers to a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, 1857-1859, (1990).

Examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The vaccine compositions may additionally include a biologically acceptable buffer to maintain a pH close to neutral (7.0-7.3). Exemplary buffers are phosphate, carboxylate, and bicarbonate buffers. Buffering agents may include sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine composition, more preferably about 0.001-1% (w/v). Other excipients, if desired, may be included as part of the final vaccine composition. The remainder of the vaccine composition may be an acceptable diluent, to 100%, including water. The vaccine composition may also be formulated as part of a water-in-oil, or oil-in-water emulsion.

The vaccine compositions may also comprise an adjuvant. An adjuvant is a substance or combination of substances used to increase the efficacy or potency of a vaccine composition or modulates the immune response to a vaccine composition. An adjuvant may accelerate, prolong or enhance antigen-specific immune responses when used in combination with a vaccine composition. Suitable adjuvants are known in the art and include, but are not limited to, threonyl muramyl dipeptide (MDP) (Byars et al., 1987), Ribi adjuvant system components (Corixa Corp., Seattle, Wash.) such as the cell wall skeleton (CWS) component, Freund's complete adjuvants, Freund's incomplete adjuvants, bacterial lipopolysaccharide (LPS; e.g., from E. coli), or a combination thereof. A variety of other well-known adjuvants may also be used with the methods and vaccine compositions of the invention, such as aluminum hydroxide, saponin, amorphous aluminum hydroxyphosphate sulfate (AAHS), aluminum hydroxide, aluminum phosphate, potassium aluminum sulfate (Alum), and combinations thereof. Cytokines (γ-IFN, GM-CSF, CSF, etc.), lymphokines, and interleukins (IL-1, IL-2, IL-3, IL-4, IL-5, IL-6, IL-7, IL-8. IL-9, IL-10, IL-11, IL-12, IL-13, IL-14, IL-15, IL-16, IL-17, 11-18, 11-19, IL-20, IL-21, and 11-22) have also been used as adjuvants and/or supplements within vaccine compositions and are contemplated to be within the scope of the present invention. For example, one or more different cytokines and/or lymphokines may be included in the vaccine compositions described herein. The vaccine compositions may include an aluminum salt, AS04, MF59, AS01B, CpG 1018, or another adjuvant that is considered to be safe for use in humans by the Centers for Disease Control and Prevention.

The vaccine composition may be separated into vials or other suitable containers. The vaccine composition may then be packaged in individual or multi-dose ampoules or be subsequently lyophilized before packaging in individual or multi-dose ampoules. The lyophilized vaccine composition may be stored for extended periods of time without loss of viability at ambient temperatures. The lyophilized vaccine may be reconstituted and administered to a patient. The term “lyophilization” or “lyophilized” refers to freezing of a material at low temperature followed by dehydration by sublimation, usually under a high vacuum. Lyophilization is also known as freeze drying. Many techniques of freezing are known in the art of lyophilization such as tray-freezing, shelf-freezing, spray-freezing, shell-freezing and liquid nitrogen immersion. Each technique will result in a different rate of freezing. Shell-freezing may be automated or manual. For example, flasks can be automatically rotated by motor driven rollers in a refrigerated bath containing alcohol, acetone, liquid nitrogen, or any other appropriate fluid. A thin coating of product is evenly frozen around the inside “shell” of a flask, permitting a greater volume of material to be safely processed during each freeze-drying run. Tray-freezing may be performed by, for example, placing the samples in lyophilizer, equilibrating 1 hr at a shelf temperature of 0° C., then cooling the shelves at 0.5° C./min to −40° C. Spray-freezing, for example, may be performed by spray-freezing into liquid, dropping by ˜20 μl droplets into liquid N2, spray-freezing into vapor over liquid, or by other techniques known in the art.

As used herein, “a polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, including DNA, RNA, ORFs, analogs and fragments thereof. The polynucleotides disclosed herein may be optimized, for example codon optimized or host cell optimized.

As used herein, the terms “proteins” and “polypeptides” are used interchangeably herein to designate a series of amino acid residues connected to the other by peptide bonds between the alpha-amino and carboxy groups of adjacent residues. The terms “protein” and “polypeptide” refer to a polymer of protein amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing. The antibodies of the present invention are polypeptides, as well the antigen-binding fragments and fragments thereof.

“Percentage of sequence identity”, “percent similarity”, or “percent identity” is determined by comparing two optimally aligned sequences over a comparison window, wherein the portion of the polynucleotide or peptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “substantial similarity” of polynucleotide or peptide sequences means that a polynucleotide or peptide comprises a sequence that has at least 75% sequence identity. Alternatively, percent identity can be any integer from 75% to 100%. More preferred embodiments include at least: 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

### Methods

In a third aspect, the disclosure provides a method of eliciting an immune response to a SARS-COV-2 spike protein. The method comprises administering the vaccine compositions described herein in an effective amount to elicit an immune response. The immune response elicited by the vaccine compositions described herein is increased compared to the immune response elicited by a mRNA or protein vaccine comprising the ancestral SARS-COV-2 spike protein not containing the D614G mutation.

The immune response to the vaccine composition described herein may be an adaptive immune response against the protein of SEQ ID NO: 1. The adaptive immune response includes a humoral immune response. A humoral immune response is mediated by extracellular antibody molecules and is typically characterized by Th2 activation, cytokine production, germ center formation, affinity maturation and memory cell production. Humoral immunity can also refer to the effector functions of an antibody, including recognition of a foreign entity, complement activation and opsonization. The humoral immune response to the vaccine composition described herein may comprise an increase in the antibody titers against SARS-COV-2 spike protein. Antibody titer is a laboratory test that measures the level of antibodies in a blood sample. The antibody level in the blood is a reflection of the body's past experience or exposure to an antigen, or something that the body does not recognize as self.

The term “administering” refers to contacting, dispensing, delivering or applying the vaccine composition to a subject by any suitable route for delivery to the desired location in the subject, including delivery by either the parenteral or oral administration, intramuscular injection, subcutaneous/intradermal injection, intravenous injection, intrathecal administration, buccal administration, transdermal delivery, topical administration, and intranasal or respiratory administration. The vaccine composition may be administered systemically or locally. In preferred embodiments, the vaccine is administered by intramuscular route, or another route with similar rapid absorption, preferably by injection, such as by needle injection.

In a fourth aspect, the disclosure provides a method of eliciting a long-term immune response against SARS-COV-2. The method comprises administering the vaccine composition described herein in an amount effective to elicit an immune response that lasts for longer than about 4 months from the time of administration. The immune response may last about 6 months, about 8 months, about 12 months, about 16 months, about 18 months, or longer from the time of administration. The method may comprise administering the vaccine composition at least two times, wherein the time between the first and second administration is at least about two weeks. The time between the first and second administration may be between about two weeks and about three weeks. Each dose comprises an adequate amount of vaccine to elicit an adaptive immune response.

The terms “effective amount” or “therapeutically effective amount” refer to an amount sufficient to effect beneficial or desirable biological and/or clinical results. The amount of vaccine that is therapeutically effective may vary depending on the particular pathogen or the condition of the subject. Appropriate dosages may be determined, for example, by extrapolation from animal studies or in clinical trials taking into account body weight of the patient, absorption rate, half-life, disease severity and the like. The vaccine compositions described herein may be given once, twice, or once followed by a series of boosters, e.g. once a month, once every other month, once every 4 months, once every 6 months, once every year, once every two years, and any range of time in between.

In a fifth aspect of the present invention, constructs are provided. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies. Constructs may include for example, the pcDNA3.1 mammalian expression vector with known 5′ and 3′ UTR restriction sites and markers.

The constructs provided herein may be prepared by methods available to those of skill in the art. Notably each of the constructs claimed are recombinant molecules and as such do not occur in nature. Generally, the nomenclature used herein and the laboratory procedures utilized in the present invention include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

The constructs provided herein may include a promoter operably linked to any one of the polynucleotides described herein. As used herein, a polynucleotide is “operably connected” or “operably linked” when it is placed into a functional relationship with a second polynucleotide sequence.

As used herein, the terms “heterologous promoter,” “promoter,” “promoter region,” or “promoter sequence” refer generally to transcriptional regulatory regions of a gene, which may be found at the 5′ or 3′ side of a polynucleotides described herein, or within the coding region of said polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

Heterologous promoters useful in the practice of the present invention include, but are not limited to, constitutive, inducible, temporally-regulated, developmentally regulated, chemically regulated, tissue-preferred and tissue-specific promoters. The heterologous promoter may be a plant, animal, bacterial, fungal, or synthetic promoter. Suitable promoters are known and described in the art. Suitable promoters include the T3, T7 and SP6 promoter sequences, which are often used for in vitro transcription of RNA. In mammalian cells, typical promoters include, without limitation, promoters for Rous sarcoma virus (RSV), human immunodeficiency virus (HIV-1), cytomegalovirus (CMV), SV40 virus, as well as the translational elongation factor EF-1α promoter or ubiquitin promoter.

Host cells capable of producing the mRNA described herein are also contemplated. Suitable host cells are known in the art, and may comprise a construct encoding the mRNA of the D614G mutant.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps.

All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All publications, patents, and patent applications mentioned herein are fully incorporated by reference, unless explicitly indicated otherwise. The present disclosure shall control in the event there are any disparities between any definitions and/or description found in the cited references. Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following examples are meant only to be illustrative and are not meant as limitations on the scope of the invention or of the appended claims.

### Example 1

### Mice and Vaccinations

6-8-week-old C57BL/6 mice from Jackson laboratories were used. Mice were housed at the Northwestern University Center for Comparative Medicine (CCM). Mice were immunized intramuscularly with mRNA-based LNP vaccine encoding SARS-COV-2 spike protein (3 ug in 100 μL PBS). 50 μL of vaccine were administered in each quadricep. mRNA vaccines were synthesized encoding for the codon-optimized SARS-COV-2 spike protein from strain USA-WA1/2020 or the D614G variant (only difference is 1 amino acid at position 614 of the spike protein, which renders the spike protein more stable) (FIG. 1). (Korber et al. 2020, Cell 182, 812-827; Zhang et al. Trends in Genetics, April 2021, Vol. 37, No. 4; Zhang et al. Science. 2021 Apr. 30, 372(6541):525-530). Constructs contained a T7 promoter site for in vitro transcription of mRNA, 5′ UTR and 3′ UTRs. mRNA was synthesized from linearized DNA with T7 in vitro transcription kits from CellScript and following manufacturer's protocol. RNA was generated with pseudouridine in place of uridine with the Incognito mRNA synthesis kit (Cat #C-ICTY110510). 5′ cap-1 structure and 3′ poly-A tail were enzymatically added. mRNA was encapsulated into lipid nanoparticles using the PNI Nanosystems NanoAssemblr Benchtop system. mRNA was dissolved in PNI Formulation Buffer (Cat #NWW0043) and was run through a laminar flow cartridge with Gen Voy ILM (Cat #NWW0041) encapsulation lipids at a flow ratio of 3:1 (RNA in PNI Buffer: Gen Voy ILM) at total flow rate of 12 mL/min to produce mRNA-LNPs. These mRNA-LNPs were characterized for encapsulation efficiency and mRNA concentration via RiboGreen Assay using Invitrogen's Quant-iT RiboGreen RNA Assay Kit (Cat #R11490).

### Protein-Specific ELISA (SARS-COV-2 Receptor Binding Domain (RBD)—Specific ELISA)

Antigen-specific total antibody titers were measured by ELISA, as follows. 96-well flat-bottom MaxiSorp plates (Thermo Scientific) were coated with 1 ug/ml of respective protein, for 48 hr at 4° C. Plates were washed three times with wash buffer (PBS+0.05% Tween 20). Blocking was performed with blocking solution (200 μl of PBS+0.05% Tween 20+2% bovine serum albumin), for 4 hr at room temperature. 6 μl of sera (plasma for human ELISAs) were added to 144 μl of blocking solution in the first column of the plate, 1:3 serial dilutions were performed until row 12 for each sample, and plates were incubated for 60 min at room temperature. Plates were washed three times with wash buffer followed by addition of secondary antibody conjugated to horseradish peroxidase, goat anti-mouse IgG (Southern Biotech) diluted in blocking solution (1:5000) at 100 μl/well were added and incubated for 60 min at room temperature. After washing plates three times with wash buffer, 100 μl/well of Sure Blue substrate (SeraCare) was added for 1 min. Reaction was stopped using 100 μl/well of KPL TMB Stop Solution (SeraCare). Absorbance was measured at 450 nm using a Spectramax Plus 384 (Molecular Devices).

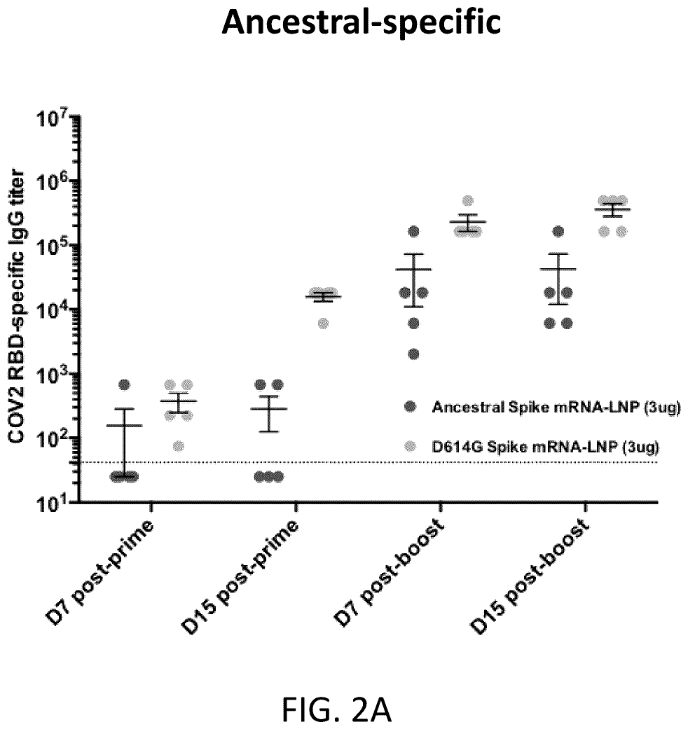

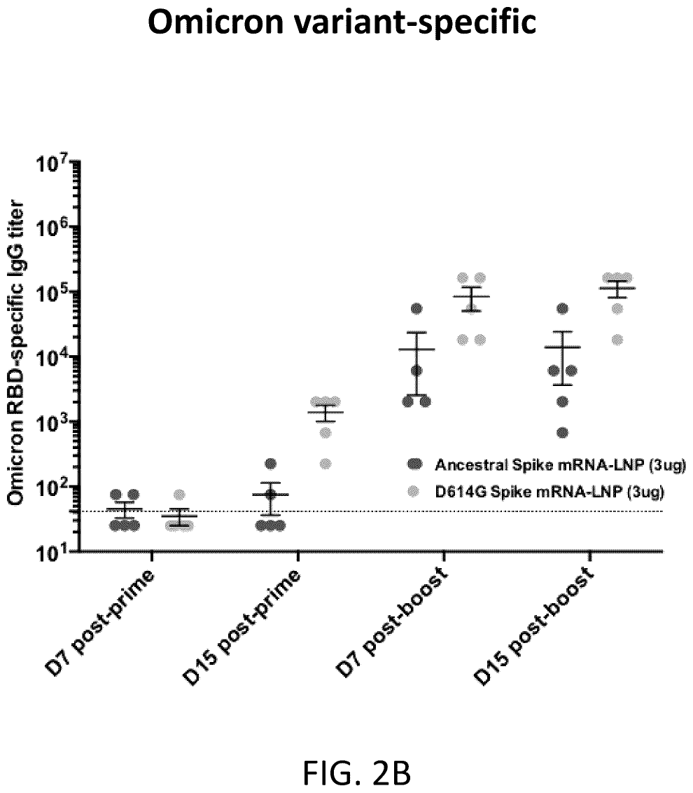

Mice immunized with D614G spike mRNA-LNP vaccine had higher SARS-COV-2 RBD specific titers compared to mice immunized with the ancestral spike mRNA vaccine as measured in a neutralization assay (FIG. 2A). The D614G vaccine elicited higher IgG titers at 15 days post-prime, as well as 7 and 15 days post-boost when challenged with ancestral-specific SARS-COV-2. Additionally, in mice challenged with Omicron variant-specific SARS-COV-2, D614G also elicited higher IgG titers at 15 days post-prime as well as 7 and 15 days post-boost (FIG. 2B). These data indicate that the D614G-based vaccine induced a more durable immune response than the currently available SARS-COV-2 mRNA vaccines.

These data suggest that vaccines comprising the structurally stable antigen D614G spike protein is more effective than antigen-matched vaccines.

| SEQUENCES | |
|---|---|
| SARS-COV-2 D614G spike protein sequence | |
| (SEQ ID NO: 1): | |
| 01 | mfvflvllpl vssqcvnltt rtqlppaytn sftrgvyypd kvfrssvlhs tqdlflpffs |
| 61 | nvtwfhaihv sgtngtkrfd npvlpfndgv yfasteksni irgwifgttl dsktqslliv |
| 121 | nnatnvvikv cefqfcndpf lgvyyhknnk swmesefrvy ssannctfey vsqpflmdle |
| 181 | gkqgnfknlr efvfknidgy fkiyskhtpi nlvrdlpqgf saleplvdlp iginitrfqt |
| 241 | llalhrsylt pgdsssgwta gaaayyvgyl qprtfllkyn engtitdavd caldplsetk |
| 301 | ctlksftvek giyqtsnfrv qptesivrfp nitnlcpfge vfnatrfasv yawnrkrisn |
| 361 | cvadysvlyn sasfstfkcy gvsptklndl cftnvyadsf virgdevrqi apgqtgkiad |
| 421 | ynyklpddft gcviawnsnn ldskvggnyn ylyrlfrksn lkpferdist eiyqagstpc |
| 481 | ngvegfncyf plqsygfqpt ngvgyqpyrv vvlsfellha patvcgpkks tnlvknkcvn |
| 541 | fnfngltgtg vltesnkkfl pfqqfgrdia dttdavrdpq tleilditpc sfggvsvitp |
| 601 | gtntsnqvav lyqgvnctev pvaihadqlt ptwrvystgs nvfqtragcl igaehvnnsy |
| 661 | ecdipigagi casyqtqtns prrarsvasq siiaytmslg aensvaysnn siaiptnfti |
| 721 | svtteilpvs mtktsvdctm yicgdstecs nlllqygsfc tqlnraltgi aveqdkntqe |
| 781 | vfaqvkqiyk tppikdfggf nfsqilpdps kpskrsfied llfnkvtlad agfikqygdc |
| 841 | 1gdiaardli caqkfngltv lpplltdemi aqytsallag titsgwtfga gaalqipfam |
| 901 | qmayrfngig vtqnvlyenq klianqfnsa igkiqdslss tasalgklqd vvnqnaqaln |
| 961 | tlvkqlssnf gaissvlndi lsrldkveae vqidrlitgr lqslqtyvtq qliraaeira |
| 1021 | sanlaatkms ecvlgqskrv dfcgkgyhlm sfpqsaphgv vflhvtyvpa qeknfttapa |
| 1081 | ichdgkahfp regvfvsngt hwfvtqrnfy epqiittdnt fvsgncdvvi givnntvydp |
| 1141 | lqpeldsfke eldkyfknht spdvdlgdis ginasvvniq keidrlneva knlneslidl |
| 1201 | qelgkyeqyi kwpwyiwlgf iagliaivmv timlccmtsc csclkgccsc gscckfdedd |
| 1261 | sepvlkgvkl hyt |
| SARS-COV-2 D614G spike protein nucleic acid sequence | |
| (SEQ ID NO: 2): | |
| 721 | ttgaaatttt attttttttt tttggaatat aaatagactc gaggccacca tgtttgtgtt |
| 781 | tcttgtttta ttgccactag tctctagtca gtgtgttaat cttacaacca gaactcaatt |
| 841 | accccctgca tacactaatt ctttcacacg tggtgtttat taccctgaca aagttttcag |
| 901 | atcctcagtt ttacattcaa ctcaggactt gttcttacct ttcttttcca atgttacttg |
| 961 | gttccatgct atacatgtct ctgggaccaa tggtactaag aggtttgata accctgtctt |
| 1021 | accatttaat gatggtgttt attttgcttc cactgagaag tctaacataa taagaggctg |
| 1081 | gatttttggt actactttag attcgaagac ccagtcctta ttaattgtta ataacgctac |
| 1141 | taatgttgtt attaaagtct gtgaatttca attttgtaat gatccatttt taggtgttta |
| 1201 | ttaccacaaa aacaacaaaa gttggatgga aagtgagttc agagtttatt ctagtgcgaa |
| 1261 | taattgcact tttgaatatg tctctcagcc ttttttaatg gaccttgaag gaaaacaggg |
| 1321 | taatttcaaa aatttaagag aatttgtgtt taagaatatt gatggttatt ttaaaatata |
| 1381 | ttctaagcac acgcctatta atttagtgag agatctccct cagggttttt cggctttaga |
| 1441 | accattggta gatttaccaa taggtattaa catcactaga tttcaaactt tattagcttt |
| 1501 | acatagaagt tatttaactc ctggtgattc ttcttcaggt tggacagctg gtgctgcagc |
| 1561 | ttattatgtg ggttatttac aacctagaac tttcttatta aaatataatg aaaatggaac |
| 1621 | cattacagat gctgtagact gtgcattaga ccctttatca gaaacaaagt gtacgttaaa |
| 1681 | atccttcact gtagaaaaag gaatctatca aacttctaac tttagagtcc aaccaacaga |
| 1741 | atctattgtt agatttccta atattacaaa cttatgccct tttggtgaag tttttaacgc |
| 1801 | caccagattt gcatctgttt atgcttggaa cagaaagaga atcagcaact gtgttgctga |
| 1861 | ttattctgtc ttatataatt ccgcatcatt ttccactttt aagtgttatg gagtgtctcc |
| 1921 | tactaaatta aatgatctct gctttactaa tgtctatgca gattcatttg taattagagg |
| 1981 | tgatgaagtc agacaaatcg ctccagggca aactggaaag attgctgatt ataattataa |
| 2041 | attaccagat gattttacag gctgcgttat agcttggaat tctaacaatt tagattctaa |
| 2101 | ggttggtggt aattataatt acttatatag attatttaga aagtctaatt taaaaccttt |
| 2161 | tgagagagat atttcaactg aaatctatca ggccggtagc acaccttgta atggtgttga |
| 2221 | aggttttaat tgttactttc ctttacaatc atatggtttc caacccacta atggtgttgg |
| 2281 | ttaccaacca tacagagtag tagtattatc ttttgaactt ttacatgcac cagcaactgt |
| 2341 | ttgtggacct aaaaagtcta ctaatttagt taaaaacaaa tgtgtcaatt tcaacttcaa |
| 2401 | tggtttaaca ggcacaggtg ttttaactga gtctaacaaa aagtttctgc ctttccaaca |
| 2461 | atttggcaga gacattgctg acactactga tgctgtcaga gatccacaga cattagagat |
| 2521 | tttagacatt acaccatgtt cttttggtgg tgtcagtgtt ataacaccag gaacaaatac |
| 2581 | ttctaaccag gttgctgttt tatatcaggg tgttaactgc acagaagtcc ctgttgctat |
| 2641 | tcatgcagat caattaactc ctacttggag agtttattct acaggttcta atgtttttca |
| 2701 | aacaagagca ggctgtttaa taggggctga acatgtcaac aactcatatg agtgtgacat |
| 2761 | acccattggt gcaggtatat gcgctagtta tcagactcag actaattctc ctgggagcgc |
| 2821 | aagtagtgta gctagtcaat ccatcattgc ctacactatg tcattaggtg cagaaaattc |
| 2881 | agttgcttac tctaataact ctattgccat acccacaaat tttactatta gtgttaccac |
| 2941 | agaaatttta ccagtgtcta tgaccaagac atcagtagat tgtacaatgt acatttgtgg |
| 3001 | tgattcaact gaatgcagca atcttttgtt gcaatatggc agtttctgta cacaattaaa |
| 3061 | ccgtgcttta actggaatag ctgttgaaca agacaaaaac acccaagaag tttttgcaca |
| 3121 | agtcaaacaa atttacaaaa caccaccaat taaagatttt ggtggtttta atttttcaca |
| 3181 | aatattacca gatccatcaa aaccaagcaa gagatcacca attgaagatt tacttttcaa |
| 3241 | caaagtgaca ttagcagatg ctggcttcat caaacaatat ggtgattgct taggtgatat |
| 3301 | tgctgctaga gacttaattt gtgcacaaaa gtttaacggc ttaactgttt taccaccttt |
| 3361 | attaacagat gaaatgattg ctcaatacac ttctgcatta ttagcgggta caatcacttc |
| 3421 | tggttggacc tttggtgcag gtccggcatt acaaatacca tttccgatgc aaatggctta |
| 3481 | tagatttaat ggtattggag ttacacagaa tgttttatat gagaaccaaa aattaattgc |
| 3541 | caaccaattt aatagtgcta ttggcaaaat tcaagactca ttatcttcca caccaagtgc |
| 3601 | attaggaaaa ttacaagatg tggtcaacca aaatgcacaa gctttaaaca cgttagttaa |
| 3661 | acaattaagc tccaattttg gtgcaatttc aagtgtttta aatgatatct tatcaagatt |
| 3721 | agacccacca gaggctgaag tgcaaattga tagattaatc acaggcagat tacaaagttt |
| 3781 | acagacatat gtgactcaac aattaattag agctgcagaa atcagagctt ctgctaattt |
| 3841 | agctgctact aaaatgtcag agtgtgtatt aggacaatca aaaagagttg atttctgtgg |
| 3901 | aaagggctat catttaatgt ccttccctca gtcagcacct catggtgtag tcttcttaca |
| 3961 | tgtgacttat gtccctgcac aagaaaagaa cttcacaact gctcctgcca tttgtcatga |
| 4021 | tggaaaagca cactttccta gagaaggtgt ctttgtttca aatggcacac actggtttgt |
| 4081 | aacacaaaga aatttctatg aaccacaaat cattactaca gacaacacat ttgtgtctgg |
| 4141 | taactgtgat gttgtaatag gaattgtcaa caacacagtt tatgatcctt tacaacctga |
| 4201 | attagactca ttcaaggagg agttagataa atattttaag aatcatacat caccagatgt |
| 4261 | tgatttaggt gacatctctg gcattaatgc ttcagttgta aacattcaaa aagaaattga |
| 4321 | cagattaaat gaggttgcca agaatttaaa tgaatcttta atcgatttac aagaattagg |
| 4381 | aaagtatgag cagtatataa aatggccatg gtacatttgg ttaggtttta tagctggctt |
| 4441 | aattgccata gtaatggtga caattatgtt atgctgtatg accagttgct gtagttgttt |
| 4501 | aaagggctgt tgttcttgtg gatcctgctg caaatttgat gaagacgact ctgagccagt |
| 4561 | gttaaaagga gtcaaattac attacacata accgtcagta tcggcggaat tacagctgag |

SARS-COV-2 D614 portion of genomic sequence
(SEQ ID NO: 3):
TATCAGGATGTTAACTGC
SARS-COV-2 G614 portion of genomic sequence
(SEQ ID NO: 4):
TATCAGGGTGTTAACTGC
SARS-COV-2 D614 portion of amino acid
(SEQ ID NO: 5):
YQDVMC
SARS-COV-2 G614 portion of amino acid
(SEQ ID NO: 6):
YQGVNC

1. A vaccine composition capable of eliciting an immune response in a subject against SARS-Cov-2, the vaccine composition comprising:
a mutant SARS-Cov-2 spike protein comprising D614G mutation (SEQ ID NO:1) or a portion thereof; or
an mRNA encoding the mutant SARS-Cov-2 spike protein or portion thereof.

2. The vaccine composition of claim 1, wherein the portion of the mutant SARS-Cov-2 spike protein comprising D614G mutation comprises SEQ ID NO: 6.

3. The vaccine composition of claim 1, further comprising a nanoparticle.

4. The vaccine composition of claim 3, wherein the nanoparticle encapsulates the protein or mRNA.

5. The vaccine composition of claim 3, wherein the nanoparticle is a lipid nanoparticle.

6. A method of eliciting an immune response to a SARS-COV-2 spike protein in a subject, the method comprising administering to the subject the vaccine composition of claim 1 in an amount effective to elicit the immune response.

7. The method of claim 6, wherein the immune response is a humoral immune response.

8. The method of claim 7, wherein the humoral immune response comprises an increase in the antibody titers against SARS-COV-2 spike protein.

9. The method of claim 6, wherein the administering comprises administering the vaccine composition at least two times.

10. The method of claim 7, wherein the time between the first and second administration is at least about two weeks.

11. The method of claim 7, wherein the time between the first and second administration is between about two and about three weeks.

12. The method of claim 6, wherein the method comprises administering the vaccine intramuscularly.

13. The method of claim 6, wherein the subject is a human.

14. A method of eliciting a long-term immune response against SARS-COV-2, the method comprising administering the vaccine of claim 1 in an amount effective to elicit the immune response for longer than six months from the time of administration.

15. The method of claim 14, wherein the administering comprises administering the vaccine at least two times.

16. The method of claim 15, wherein the time between the first and second administration is at least about two weeks.

17. The method of claim 16, wherein the time between the first and second administration is between about two and about three weeks.

18. The method of claim 14, wherein the immune response lasts longer than about 12 months from the time of administration.

19. The method of claim 14, wherein the method comprises administering the vaccine intramuscularly.

20. The method of claim 14, wherein the subject is a human.


SEQUENCE LISTING (6 sequences)

SEQ ID NO: 1 - protein, 1273 aa, synthetic construct
  MUTAGEN 614: note = D614G
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3900 bp, synthetic construct
  source 1..3900: mol_type = other DNA
  variation 1890: note = D614G
ttgaaattttattttttttttttggaatataaatagactcgaggccaccatgtttgtgtttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcttaccatttaatgatggtgtttattttgcttccactgagaagtctaacataataagaggctggatttttggtactactttagattcgaagacccagtccttattaattgttaataacgctactaatgttgttattaaagtctgtgaatttcaattttgtaatgatccatttttaggtgtttattaccacaaaaacaacaaaagttggatggaaagtgagttcagagtttattctagtgcgaataattgcacttttgaatatgtctctcagccttttttaatggaccttgaaggaaaacagggtaatttcaaaaatttaagagaatttgtgtttaagaatattgatggttattttaaaatatattctaagcacacgcctattaatttagtgagagatctccctcagggtttttcggctttagaaccattggtagatttaccaataggtattaacatcactagatttcaaactttattagctttacatagaagttatttaactcctggtgattcttcttcaggttggacagctggtgctgcagcttattatgtgggttatttacaacctagaactttcttattaaaatataatgaaaatggaaccattacagatgctgtagactgtgcattagaccctttatcagaaacaaagtgtacgttaaaatccttcactgtagaaaaaggaatctatcaaacttctaactttagagtccaaccaacagaatctattgttagatttcctaatattacaaacttatgcccttttggtgaagtttttaacgccaccagatttgcatctgtttatgcttggaacagaaagagaatcagcaactgtgttgctgattattctgtcttatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatttagattctaaggttggtggtaattataattacttatatagattatttagaaagtctaatttaaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtattatcttttgaacttttacatgcaccagcaactgtttgtggacctaaaaagtctactaatttagttaaaaacaaatgtgtcaatttcaacttcaatggtttaacaggcacaggtgttttaactgagtctaacaaaaagtttctgcctttccaacaatttggcagagacattgctgacactactgatgctgtcagagatccacagacattagagattttagacattacaccatgttcttttggtggtgtcagtgttataacaccaggaacaaatacttctaaccaggttgctgttttatatcagggtgttaactgcacagaagtccctgttgctattcatgcagatcaattaactcctacttggagagtttattctacaggttctaatgtttttcaaacaagagcaggctgtttaataggggctgaacatgtcaacaactcatatgagtgtgacatacccattggtgcaggtatatgcgctagttatcagactcagactaattctcctgggagcgcaagtagtgtagctagtcaatccatcattgcctacactatgtcattaggtgcagaaaattcagttgcttactctaataactctattgccatacccacaaattttactattagtgttaccacagaaattttaccagtgtctatgaccaagacatcagtagattgtacaatgtacatttgtggtgattcaactgaatgcagcaatcttttgttgcaatatggcagtttctgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagagatcaccaattgaagatttacttttcaacaaagtgacattagcagatgctggcttcatcaaacaatatggtgattgcttaggtgatattgctgctagagacttaatttgtgcacaaaagtttaacggcttaactgttttaccacctttattaacagatgaaatgattgctcaatacacttctgcattattagcgggtacaatcacttctggttggacctttggtgcaggtccggcattacaaataccatttccgatgcaaatggcttatagatttaatggtattggagttacacagaatgttttatatgagaaccaaaaattaattgccaaccaatttaatagtgctattggcaaaattcaagactcattatcttccacaccaagtgcattaggaaaattacaagatgtggtcaaccaaaatgcacaagctttaaacacgttagttaaacaattaagctccaattttggtgcaatttcaagtgttttaaatgatatcttatcaagattagacccaccagaggctgaagtgcaaattgatagattaatcacaggcagattacaaagtttacagacatatgtgactcaacaattaattagagctgcagaaatcagagcttctgctaatttagctgctactaaaatgtcagagtgtgtattaggacaatcaaaaagagttgatttctgtggaaagggctatcatttaatgtccttccctcagtcagcacctcatggtgtagtcttcttacatgtgacttatgtccctgcacaagaaaagaacttcacaactgctcctgccatttgtcatgatggaaaagcacactttcctagagaaggtgtctttgtttcaaatggcacacactggtttgtaacacaaagaaatttctatgaaccacaaatcattactacagacaacacatttgtgtctggtaactgtgatgttgtaataggaattgtcaacaacacagtttatgatcctttacaacctgaattagactcattcaaggaggagttagataaatattttaagaatcatacatcaccagatgttgatttaggtgacatctctggcattaatgcttcagttgtaaacattcaaaaagaaattgacagattaaatgaggttgccaagaatttaaatgaatctttaatcgatttacaagaattaggaaagtatgagcagtatataaaatggccatggtacatttggttaggttttatagctggcttaattgccatagtaatggtgacaattatgttatgctgtatgaccagttgctgtagttgtttaaagggctgttgttcttgtggatcctgctgcaaatttgatgaagacgactctgagccagtgttaaaaggagtcaaattacattacacataaccgtcagtatcggcggaattacagctgag

SEQ ID NO: 3 - DNA, 18 bp, synthetic construct
  source 1..18: mol_type = other DNA
tatcaggatgttaactgc

SEQ ID NO: 4 - DNA, 18 bp, synthetic construct
  source 1..18: mol_type = other DNA
tatcagggtgttaactgc

SEQ ID NO: 5 - protein, 6 aa, synthetic construct
YQDVMC

SEQ ID NO: 6 - protein, 6 aa, synthetic construct
YQGVNC